(19) United States
(12) Patent Application Publication
(10) Pub. No.: US 2013/0296995 A1
(43) Pub. Date: Nov. 7, 2013

(54) Method For Treating A Tissue Region With An Electric Field

(71) Applicants: Faisal Mahmood, Ishoj (DK); Lasse G. Staal, Jyllinge (DK); Karen Julie Gehl, Vanlose (DK); Birgit Agerholm Larsen, Birkerod (DK)

(72) Inventors: Faisal Mahmood, Ishoj (DK); Lasse G. Staal, Jyllinge (DK); Karen Julie Gehl, Vanlose (DK); Birgit Agerholm Larsen, Birkerod (DK)

(73) Assignees: Glostrup Hospital, Glostrup (DK); Herlev Hospital, Herlev (DK)

(21) Appl. No.: 13/880,947

(22) PCT Filed: Oct. 21, 2011
(86) PCT No.: PCT/EP2011/068432

§ 371 Date: Jul. 16, 2013

Related U.S. Application Data
(60) Provisional application No. 61/405,954, filed on Oct. 22, 2010.

(51) Int. Cl. A61N 1/04

(52) U.S. Cl. CPC A61N 1/0416
USPC 607/148

(57) ABSTRACT

A method for treating a patient by covering a tissue region with a electric field capable of transiently or permanently permeabilizing cells in said tissue region, comprising the steps of: a. positioning the electrodes of an electrode device in a tissue region to be treated; and b. creating a specific polarity pattern by applying to a first subset of electrodes comprising at least two electrodes a first polarity while at the same time applying to a second subset of electrodes comprising at least two electrodes a second polarity.

### FIELD OF THE INVENTION

The present invention relates to a method of treating a tissue region with an electric field.

### BACKGROUND OF THE INVENTION

Malignant brain cancer is a disease with a grim prognosis. In spite of several decades of research into improved treatment methods, 5-year survival rates are still low, with some cancer types presenting with a 5-year survival of less than 10%.

Electrochemotherapy is a novel cancer treatment methodology that has shown strong performance in the treatment of superficial tumors. The basic concept is that the application of an electric field with specific parameters to a target tissue will cause cells in that target region to become porous. As a result, diffusion of extraneous material across the cell membrane may be strongly amplified, and the cytotoxic effect of certain drugs may be amplified hundred-to thousand-fold as their access to the cytosol is increased. However, the lack of electrode devices capable of applying an electric field to deeper-lying tissue regions has prevented the use of electrochemotherapy in the treatment of patients suffering from brain cancer or other deep-seated tumors.

### ASPECTS OF THE INVENTION

In a FIRST aspect, the present invention relates to a method for treating a patient by covering a tissue region with a electric field capable of transiently or permanently permeabilizing cells in said tissue region, comprising the steps of:
a. positioning the electrodes of an electrode device in a tissue region to be treated
b. creating a specific polarity pattern by applying to a first subset of electrodes comprising at least two electrodes a first polarity while at the same time applying to a second subset of electrodes comprising at least two electrodes a second polarity.

In a SECOND aspect the present invention relates to a treatment system for the treatment of cancers and other diseases, the system being adapted to carry out the method according to the first aspect of the invention, the treatment system comprising a pulse generating device, a switching device and an electrode device with a symmetry axis, wherein the devices are in operative connection; wherein the electrode device comprises at least two electrodes; wherein the switching device is adapted to assign a specific polarity to each of the electrodes; wherein the switching device is adapted to activate and deactivate the electrodes so as to define a treatment volume of variable dimensions and geometry.

In a THIRD aspect the present invention relates to a method for treating a patient according the invention according to the first and/or the second aspect of the invention, further comprising the step of mapping the tissue region to be treated before initiate the treatment

### DESCRIPTION OF THE INVENTION

In the remaining part of this document, the invention according to the first, second and the third aspect is described in further detail.

To solve the problems mentioned under the background of the invention and other problems, a novel treatment system including an electrode device for electrotransfer of anti-neoplastic drugs and genes to intracranial tumors in humans is presented, along with methods for the optimization of electrode devices for intracranial and other applications.

A specific embodiment of an electrode device has been developed and optimized using a numerical model to determine the electric field distribution in an idealized spherical target volume.

A semiempirical objective function that imposes treatment constrains parallel to those known from radiation therapy has been used for scoring variations of the device geometries.

In addition, the geometrical tolerances of the system have been assessed in terms of tolerated deflection of the electrodes and device positioning inaccuracy. The results show that small geometrical changes may yield significant improvement from a base-line design. E.g. 2 mm displacement of 6 electrodes yields 14% better compliance with the clinical parameters, compared to the base-line design (prototype), and additionally makes the electrode device less sensitive to random geometrical deviations. The feasibility of the optimization method is readily applicable to other electrode configurations, as will be understood by those skilled in the art.

Furthermore, advantageous methods of using said treatment system and said electrode device in the treatment of patients are disclosed.

The electrode device may be highly customizable and may be been developed to offer a high degree of treatment flexibility, which may be exploited in several ways.

An advantageous characteristic of the electrode device may by that it offers the physician the option of changing electric field shape and size during treatment. This characteristic of the electrode device may be known as “flexible deployment”. A specific embodiment of the electrode device comprises multiple electrode length settings (two or more), enabling the physician to choose between shorter and longer deployment lengths. In said specific embodiment, shorter deployment lengths may be associated with electric fields having a smaller diameter than electric fields that will result from longer deployment lengths. It will be evident to those skilled in the art that other forms of flexible deployment will follow naturally from the exploitation of the concepts presented in this document, and that these are encompassed by the scope of this application.

Alternatively, the device itself may be customized in the production to suit specific treatment requirements. Most notably, trajectories and lengths of electrodes may be changed to suit different anatomies. A standard geometry and means for establishing said geometry is proposed, but it will be evident to those skilled in the art that other geometries will follow naturally from the exploitation of the concepts presented in this document, and that these are encompassed by the scope of this application.

Electric field strength may also be changed through the changing of pulse parameters on a pulse generator. This may for instance be of value when an electrode device comprising flexible deployment is used, and where treatments employing shorter deployment lengths and resulting smaller-diameter fields may require les field strength than treatments employing longer deployment lengths and resulting larger-diameter fields. A standard pulse protocol, including standard field strengths, is proposed, but it will be evident to those skilled in the art that other protocols will follow naturally from the exploitation of the concepts presented in this document, and that these are encompassed by the scope of this application.

A particular advantage of the electrode device proposed in this document is that it comprises electrodes that may be individually and selectively activated as part of treatment preparation and/or treatment execution. Such selective activation and/or deactivation may for instance be accomplished by means of a switching or routing device with a programmable interface that may form part of the treatment system. Programming may for instance be accomplished by means of a laptop that may be connected to the switching or routing device. Alternatively, the device may comprise an integrated interface enabling the physician to communicate his intent to the device.

This principle may be exploited in the treatment of tumors with non-spherical geometries, where selective deactivation of electrodes that are placed in healthy tissue may result in a sparing of said tissue.

A particularly advantageous application of this principle for the treatment of diseases in the brain is the preoperative interrogation—or mapping—of tissue in the target tissue region. Such an interrogation may be used in open-skull surgery to avoid damage to brain regions that are critical to patient functioning, but no other treatment modality offers the physician the option of determining in advance whether treatment is safe or not.

Interrogation may be done by applying a test current, that may e.g. be 20 mA, delivered by a constant-current stimulator that may for instance be integrated in the switching or routing device. Programming of the test current application patterns may for instance be done through the laptop interface. Test current may be delivered between pairs or groups of electrodes with the patient awake. Based on the response to these test currents, the physician may subsequently decide to turn off electrodes that have been found to cause suppression of normal patient functions, for instance through the programmable interface of the switching or routing device. Thus, destruction of tissue regions that are critical to patient functioning may be avoided.

Such deactivation of electrodes following insertion of the device into the brain may require changes in the treatment plan, most notably the pulse sequences delivered to the remaining electrodes. Current state-of-the-art pulse generators offer limited options of adapting a treatment plan to take into consideration e.g. changes in the electrode device configuration or tissue characteristics. This document, in disclosing an optimization methodology and a treatment system enabling the implementation of this methodology, provides a framework whereby a physician may rapidly adapt a given treatment plan to changing circumstances, thus providing increased flexibility in the treatment planning to the benefit of the patient.

Finally, it may be advantageous to be able to determine, before the treatment commences, whether short-circuits have occurred between electrodes that are to have opposing polarities. While it is possible to map the positions of individual electrodes after placement of the electrode device in the patient's brain, it may often be desirable to avoid this costly addition to the treatment. Instead, test voltages may be applied between pairs of electrodes to determine whether electrodes have been deflected outside the permissible limits.

### Further Information

When biological tissue is exposed to an excessive electrical force the phospho-lipid bi-layer of the cell membranes in the tissue may be focally permeabilized allowing transport of extracellular polar molecules into the cytoplasm. This procedure is in the context of the present invention called electropermeabilization or electroporation (EP) and its use in gene electrotransfer in mammalian cells was shown in 1982 [1]. Gene electrotransfer refers to EP mediated transport of DNA into the cells and it has already demonstrated its potential as a means of gene therapy of cancer [2, 3]. Recently (early 1990ies) a technique termed electrochemotherapy (ECT) was invented proving the potentiation of antitumor effect of chemotherapeutics by applying local electrical pulses [4, 5]. Notably, the cytotoxicity of bleomycin may be enhanced over 300 fold with ECT [4, 6]. However, for EP to occur the intensity of the local electric field must surpass a certain tissue specific threshold Erev. Reversible EP is found to last for minutes at physiological temperature, depending on the tissue and the electric pulse parameters, after which the cells regain molecular homeostasis [8, 9].

If the applied electric field becomes yet stronger, exceeding a value Eirrev, the changes induced in the phospho-lipid layers are more pronounced and the cells eventually die due to prolonged adverse ion concentrations [7]. This effect may be exploited, e.g. in the treatment of cancerous tissue, but since the challenge of optimization is considered more pronounced with applications based on reversible electroporation, ECT will be the topic for the rest of this document. Those skilled in the art will recognize that the principles disclosed herein will be applicable also to irreversible electroporation.

So far ECT has been exploited for clinical targets such as cutaneous metastases from disseminated malignant melanoma, breast cancer, head and neck cancer [10-14]. Current technology has limited applicability for deeper seated-tumors. The challenge is mainly associated with placing the electrodes accurately and non-destructively to generate an electric field with the desired strength at the desired site. Another challenge concerns the delivery of the correct electric field intensity to the target site and at the same time sparing normal tissue in its close vicinity. Attempts to overcome these obstacles are not only advisable [15, 16], but essential for putting ECT forward as a real alternative to e.g. palliative external beam radiation therapy (EBRT). EBRT may be fundamentally limited since irradiation of deep-seated tumors involves traversal of normal tissue, inducing normal tissue toxicity. Due to this, recurrence of tumor at the irradiated target site may be frequent, indicative of the dose at the tumor site being too low. ECT may in principle not limited by these issues and may be capable of delivering more conformal treatments, if the target tissue is made accessible. For instance, the ability of EP to interact with the tissue before treatment, enabling the mapping—or identification—of critical structures before treatment is unique and compares favorably with EBRT.

The electric field in the target tissue is influenced by several factors: the electrical properties of the tissue, the electrical pulse amplitude and the geometry of the electrodes. The geometry of the electrode device and the applied voltage are adjustable factors, whereas the tissue property is not. Thus manipulation of the electric field may be handled by geometrical alterations of the electrode device and/or changing the electrical potential of the electrodes.

### Geometrical Alterations

The electrode device described in this application may be characterized by offering a high degree of geometrical flexibility due to a modular design. Features increasing the flexibility of the electrode device include the following:
1. Electrodes that may be moved or deployed from a first retracted position inside an introducer system to at least one advanced position where electrodes extend into the tissue region to be treated. A particular embodiment offers the physician four different advanced positions or deployment lengths
2. Electrodes that may be individually movable between a first retracted position inside an introducer and at least one advanced position where electrodes extend into the tissue region to be treated.
3. A device tip comprising electrode channels with specific trajectories. These channels impose on the electrodes trajectories through the tissue to be treated and may be changed to suit specific tumor geometries. In a specific embodiment, electrodes are deflected away from a symmetry axis of the device, which results in a cone-shaped geometry with the apex at the tip of the electrode device. This specific embodiment may furthermore be characterized by having electrodes that are disposed in two concentric rings about a center electrode, as further described below. When a cone-shaped geometry is combined with electrodes that may be moved between different deployment lengths, coverage of tumors of different sizes may be enabled. A particular embodiment provides the physician with the option of treating tumors that are between 20 and 30 mm in diameter.
4. Electrodes that may have different characteristics, including:
   a. Materials with higher or lower stiffness, providing either increased penetration capabilities or better deflection capabilities. In a particular embodiment, electrodes are made of 35N LT, an alloy with a very high modulus of elasticity and excellent penetration characteristics
   b. Materials that are partially or fully coated with non-conductive coatings to render specific electrode regions conductive or non-conductive. These conductive regions may be of different lengths, and designs are envisioned that have variable conductive areas. In a particular embodiment, electrodes are coated with Parylene C—a strong insulator that provides break-down strengths of more than 200 V/μm.
   c. Materials that have memo-shape capabilities, enabling the creation of electrode geometries that may otherwise be impossible due to the constraints of normal metals. An alternative embodiment comprises electrodes made of Nitinol, a material characterized by high deflectability and acceptable stiffness
   d. Materials that may be MR-compatible. Electrodes made of 35N LT or Nitinol share these characteristics
   e. Materials that are biocompatible. Electrodes made of 35N LT or Nitinol share these characteristics
   f. A variety of shapes, including different shapes of distal tips and electrode bodies and different lengths. A particular embodiment comprises cylindrical electrodes with rounded distal ends while another embodiment comprises flattened electrodes with sharpened distal ends. Another advantageous embodiment comprises electrodes that are tubular and may act as drug delivery channels.
5. Introducers featuring various numbers of electrodes with various lengths.

A particular embodiment comprises thirteen electrodes with uninsulated distal ends. These uninsulated ends have a length of 35 mm, enabling the coverage of a tumor with a diameter of 30 mm.

### Changing the Electric Field

In current state-of-the-art pulse generators, changing the parameters of the electric field means adjusting the output of the voltage generator (i.e. the duration, number and/or amplitude of pulses). However, these pulse generators are limited in their ability to adapt to changes in the treatment plan that may for instance result from the mapping of a treatment area to determine critical structures.

The simulation of the electric field resulting from a given electrode device and polarity pattern is in practice only possible using computer Models (for example based on the Finite Element Method) that handle both the applied voltage/geometry and the tissue properties (if they are known). The use of commercial software packages is the preferred way of calculation due to speed and possibility of simulating multiple physical conditions, and the integration of such software packages in future generations of pulse generators is highly desirable.

However, important drawbacks limit the stand-alone usefulness of such commercially available software packages. For one, while it may be possible to simulate the field resulting from a given geometry and polarity pattern, options for optimizing that particular geometry and polarity pattern will not be readily apparent. Further adding to the challenge of treatment planning, the complexity of the biological system still limits the validity of the electric field models. E.g. under steady state conditions the electric field intensity is proportional to the applied voltage, but experiments suggest that the resistivity of the tissue changes under the influence of electrical pulses and therefore the proportionality no longer holds [19, 20].

The method disclosed in this document may be readily applicable in a clinical setting, even without the integration of commercially available software packages into a pulse generator. Thus, the modeling of an alternative treatment algorithm—e.g. after having determined that the presence of a critical tissue region demands the switching-off of two electrodes—requires only a standard laptop such as the one that is already used in the programming of the switching or routing device. In a clinical setting/treatment situation—where rapid results are needed—modeling of new treatment plans may be considerably simplified for instance by providing a catalogue containing frequently encountered alternative configurations where various numbers of electrodes have been turned off. Such a catalogue may be built in advance using the optimization method, and will provide the physician with immediately accessible alternatives to the baseline geometry.

In the following, a novel treatment system including a clinical electroporation device (electrode device) for treatment of deep-seated tumors in soft tissue, in particular brain metastases, is described. Furthermore, a simple numerical design optimization imposing basic principles from radiation therapy is described. The objective of the optimization provided as an example of this optimization method may be to improve the geometry of the initial baseline design (prototype) to achieve improved clinical performance, but applications of variants of the disclosed method are also presented. Finally, the geometrical robustness of the device and the size of the maximum treatable volume are assessed.

Others have previously presented optimizations [17, 21]. However, little attention has been given to the clinical relevance of the optimization parameters. In this document, a specific method for optimizing the design of an electrode device is disclosed. This method is based on five independent clinical parameters and enables the assessment of performance of different iterations of a given electrode device: target tissue coverage, homogeneity of the electric field, average of the electric field intensity, high intensity regions (hot spots) and normal tissue involvement. The optimization may basically be conducted as this: 1) application of incremental geometrical changes to baseline electrode device design to yield different geometries, 2) simulation of the corresponding electric field distributions, 3) assessment of the quality of the induced electric fields in the target tissue in terms of the clinical parameters, 4) collection of the clinical parameters into a mathematical function (the objective function) and identification of the optimal geometry. In addition, the optimization data may be used to determine the robustness of the electrode devices in terms of permissible bending of the electrodes when introduced in the tissue. The error associated with the positioning of the electrode device, and how this might affect the upper limit of the treatable target volume, is also addressed. The result may bea specific optimized geometry of a baseline design, in this case an electrode device with 13 electrodes that are given specific positions. Other applications, for instance in the identification of optimal polarity patterns in case one or more of the 13 electrodes will have to be turned off as a result of a brain mapping procedure, are obvious extensions of this concept.

In the specific embodiment disclosed in this document, the objective function may be computed with high spatial resolution (small voxels) to ensure high sensitivity to the incremental differences in the device geometries. The pulse amplitude may be fixed at the maximum output of 1000 volts, to allow largest possible expansion of the electric field whilst aiming at reversible EP. EP thresholds that are empirically confirmed by rat experiments conducted by the inventor's group [22] using miniature versions of the prototype introduced in the present document are used. These threshold levels are found to be Erev=350 V/cm and Eirrev=1200 V/cm in agreement with previously reported/used values [20, 23]. The target volume may be defined as a sphere (30 mm in diameter) for simplicity. Metastatic brain tumors are generally small spherical structures and the simulation may be therefore quite close to the clinical scenario.

In this document, advantage is taken of the parallels between EBRT and ECT. For example the electric field intensity in ECT may be used analogously to the energy imparted by radiation (absorbed dose) in EBRT. Also, the tissue of the treated area may be divided into different volumes according to the level of involvement and whether it is normal tissue, sub clinical disease or known disease. The concept of applying a geometrical treatment margin, i.e. expansion of the clinical target volume (CTV) into a planning target volume (PTV) to account for geometrical uncertainties, is also adopted and used to advocate the need of a similar quantity in the ECT treatment planning. In the following particular embodiments of the invention according to the first aspect are described:

Embodiment one relates to a method for treating a patient by covering a tissue region with a electric field capable of transiently or permanently permeabilizing cells in said tissue region, comprising the steps of:
a) Positioning the electrodes of an electrode device in a tissue region to be treated;
b) Creating a specific polarity pattern by applying to a first subset of electrodes comprising at least two electrodes a first polarity while at the same time applying to a second subset of electrodes comprising at least two electrodes a second polarity.

Embodiment two relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes.

Embodiment three relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system.

Embodiment four relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system, and
c) Repeating step b). at least once.

Embodiment five relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Repeating step b). at least once, and
d) Repeating step c). rotating the polarity pattern either 60, 120, 180, 240 or 300 degrees relative to the center of the polar coordinate system.

Embodiment six relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system, and
c) Repeating step b) 5 times in such a way that the polarity pattern has been rotated 60, 120, 180, 240 and 300 degrees relative to the center of the polar coordinate system.

Embodiment seven relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system, and
c) Repeating step b) 5 times in such a way that the polarity pattern has been rotated 60, 120, 180, 240 and 300 degrees relative to the center of the polar coordinate system
d) Repeating step c) at least once.

Embodiment eight relates to a method for treating a patient by covering a tissue region with a electric field capable of transiently or permanently permeabilizing cells in said tissue region, comprising the steps of
a) Positioning the electrodes of said electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system, and
c) Reversing said application of polarities at least once

Embodiment nine relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities at least once, and
d) Repeating steps b) to c) while rotating the polarity pattern either 60, 120, 180, 240 or 300 degrees relative to said polar coordinate system.

Embodiment ten relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities at least once, and
d) Repeating step b) to c), sequentially rotating the polarity pattern either 60, 120, 180, 240 and 300 degrees between each repetition relative to said polar coordinate system.

Embodiment eleven relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities at least once,
d) Rotating the polarity pattern either 60, 120, 180, 240 or 300 degrees relative to said polar coordinate system, and
e) Repeating steps c) to d) in any order so that at least one polarity reversal has been applied to at least one polarity pattern.

Embodiment twelve relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities at least once,
d) Rotating the polarity pattern either 60, 120, 180, 240 or 300 degrees relative to said polar coordinate system, and
e) Repeating steps c) to d) in any order so that at least one polarity reversal has been applied to each polarity pattern.

Embodiment thirteen relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities,
d) Rotating the polarity pattern either 60, 120, 180, 240 or 300 degrees relative to said polar coordinate system, and
e) Repeating steps c) to d) in any order so that at least one polarity reversal has been applied to at least one polarity pattern and that all the polarity patterns have had an equal number of polarity reversals applied.

Embodiment fourteen relates to a method for treating a patient by covering a tissue region with a electric field capable of transiently or permanently permeabilizing cells in said tissue region, comprising the steps of
a) Positioning the electrodes of an electrode device in a tissue region to be treated
b) Creating a specific polarity pattern by applying to a first subset of electrodes comprising at least two electrodes a first polarity while at the same time applying to a second subset of electrodes comprising at least two electrodes a second polarity
said first subset of electrodes comprising five electrodes and said second subset of electrodes comprising eight electrodes, and wherein the electrodes are arranged relative to a polar coordinate system such that
   the electrodes in the first subset are arranged at the angular positions 210, 240, 270, 300 and 330 degrees in the polar coordinate system,
   the electrodes in the second subset are arranged at the angular positions 0, 30, 60, 90, 120, 150 and 180 degrees in the polar coordinate system, and
   a center-electrode is arranged in the center of the polar coordinate system such that the center-electrode defines a normal to the polar coordinate system
c) Reversing said application of polarities,
d) Rotating the polarity pattern either 60, 120, 180, 240 or 300 degrees relative to said polar coordinate system, and
e) Repeating steps c) to d) in any order so that at least one polarity reversal has been applied to each polarity pattern and that all the polarity patterns have had an equal number of polarity reversals applied.

In the following particular embodiments of the invention according to the second aspect are described:

Embodiment fifteen relates to a treatment system for the treatment of cancers and other diseases, the system being adapted to carry out the method according to the first aspect of the invention (i.e. according to any of the above embodiments the claims one to fourteen), the treatment system comprising a pulse generating device, a switching device and an electrode device with a symmetry axis, wherein the devices are in operative connection; wherein the electrode device comprises at least two electrodes; wherein the switching device is adapted to assign a specific polarity to each of the electrodes; wherein the switching device is adapted to activate and deactivate the electrodes so as to define a treatment volume of variable dimensions and geometry.

Embodiment sixteen relates to a treatment system in accordance with embodiment fifteen, wherein the electrode device comprises at least 8 electrodes.

Embodiment seventeen relates to a treatment system in accordance with any of embodiment fifteen to sixteen, wherein the electrode device comprises at least 13 electrodes.

Embodiment eighteen relates to a treatment system in accordance with any of embodiments fifteen to seventeen, where a first electrode C is co-aligned with the symmetry axis of the electrode device.

Embodiment nineteen relates to a treatment system in accordance with any of embodiments fifteen to eighteen, where said first electrode C is defined as the origo in a coordinate system, when said electrode device is viewed along said symmetry axis

Embodiment twenty relates to a treatment system in accordance with embodiments fifteen to nineteen, where at least some of said electrodes are non-parallel

Embodiment twenty-one relates to a treatment system in accordance with embodiments fifteen to twenty, wherein each electrode defines a distal tip and wherein the electrodes define a first and a second group of electrodes, wherein the distal tip of each of the electrodes in the first group defines a first circle having a center which coincides with the axis of symmetry; and wherein the distal tip of each of the electrodes in the second group defines a second circle which has a center which coincides with the axis of symmetry and wherein the axis of symmetry defines a normal to the plane defines by each of the first and the second circle.

Embodiment twenty-two relates to a treatment system according to embodiment twenty-one, wherein the electrodes of the first group defines a first cone (cone A) by extending along the sides of the first cone and such that the distal tip of the electrodes terminates at the base of the first cone, and wherein the electrodes of the second group defines a second cone (cone B) by extending along the sides of the second cone and such that the distal tip of the electrodes terminates at the base of the first cone.

Embodiment twenty-three relates to a treatment system according to any of embodiments twenty-one and twenty-two, wherein the first circle is concentric with the second circle.

Embodiment twenty-four relates to a treatment system according to any of embodiments twenty-one to twenty-three, wherein the first and second circles are concentric, having centers coinciding with electrode C

Embodiment twenty-five relates to a treatment system according to any of embodiments twenty-one to twenty-three, wherein the radius of the first circle is larger than the radius of the second circle.

Embodiment twenty-six relates to a treatment system in accordance with embodiments fifteen to twenty-five, wherein the electrodes of the first group are equidistantly distributed along the circumference of the first circle, and wherein the electrodes of the second group are equidistantly distributed along the circumference of the second circle.

Embodiment twenty-seven relates to a treatment system in accordance with embodiments fifteen to twenty-six, wherein the first group of electrodes comprises 6 electrodes, and wherein the second group of electrodes comprises 6 electrodes.

Embodiment twenty-eight relates to a treatment system in accordance with embodiments fifteen to twenty-seven, wherein the electrodes of the first group are distributed along the first circle such that the electrodes are provided at the positions 0, 60, 120, 180, 240, 300 degrees in a polar coordinate system which is provided in the plane of the first circle and which has its center at the center of the first circle, and wherein the electrodes of the second group are distributed along the second circle such that the electrodes are provided at the positions 30, 90, 150, 210, 270 and 330 degrees in a polar coordinate system which is provided in the plane of the first circle and which has its center at the center of the first circle.

In the following particular embodiments of the invention according to the third aspect are described:

Embodiment twenty-nine relates to a method for treating a patient according any of embodiments one to twenty-eight, further comprising the step of mapping the tissue region to be treated before initiate the treatment Embodiment thirty relates to a method in accordance with embodiment twenty-nine, where mapping is done by applying to pairs of electrodes a test current and monitoring tissue or patient response to said test current application

Embodiment thirty-one relates to a method in accordance with any of embodiments twenty-nine to twenty-nine, where particular tissue areas within the tissue region to be treated are excluded from treatment based on the results of the mapping

Embodiment thirty-two relates to a method in accordance with any of embodiments twenty-nine to thirty-one, where exclusion of treatment of a particular tissue area within the tissue region treated is done by deactivating those electrodes placed in the particular tissue areas, resulting in the creation a new treatment volume

Embodiment thirty-three relates to a method in accordance with any of embodiments twenty-nine to thirty-two, where the continued capability of the electric field to create transient or permanent permeabilization of the tissue in the new treatment volume is ensured by means of a reconfiguration of the polarity pattern to suit the new treatment volume.

Embodiment thirty-four relates to a method in accordance with any of embodiments twenty-nine to thirty-three, where the continued capability of the electric field to create transient or permanent permeabilization of the tissue in the new treatment volume is ensured by means of a reconfiguration of the polarity pattern, said reconfiguration being based on the matching of the profile of the treatment volume with a catalogue of pre-established treatment volumes and corresponding polarity patterns

### BRIEF DESCRIPTION OF THE FIGURES

FIG. 1. Schematic illustration of the different volume designations;

FIG. 2A. Schematic side view of the electrode device;

FIG. 2B Schematic top view of the electrode device;

FIG. 3. Example of coordinates for an individual electrode, of ring a and b respectively;

FIG. 4. Shows the electrode device in different deployment positions;

FIG. 5. Schematically shows consecutive steps of deployment of the electrode device during in a treatment situation;

FIG. 6. Table showing relations between desired deployment length and required potential differences for a specific embodiment;

FIG. 7: Shows combinations of an employed electrode polarity (upper part) and an iso-field plot (lower part) in progression;

FIG. 8. Shows electrode deflection depending on the anisotropy; and

FIG. 9. Shows an example of the objective function scores shown in grey scale.

### DETAILED DESCRIPTION OF THE FIGURES

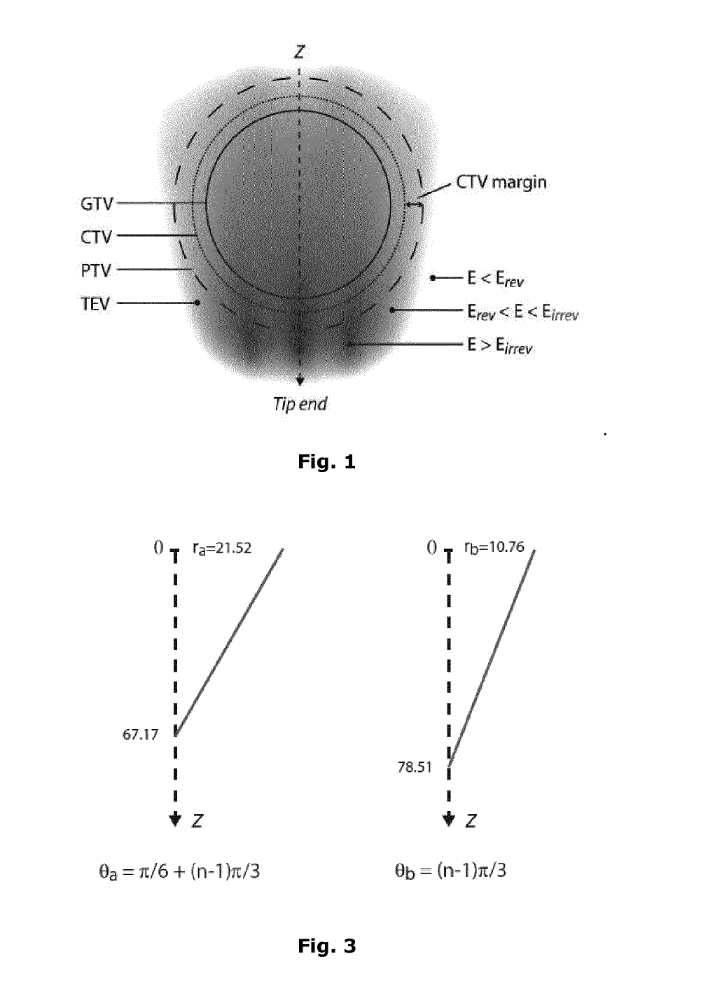

FIG. 1 shows a schematic illustration of the different volume designations. The volumes GTV, CTV, PTV are superimposed on the cross section of the 3D electric field distribution expanded by the prototype. The nomenclature used to denote the tissue involved in the EP treatment (see FIG. 1) is inspired by the definitions proposed by the International Commission on Radiation Units and Measurements (ICRU) [24]. The ICRU volume definitions have been used in EBRT for many years and form the basis of all modern treatment planning in radiation therapy.

GTV Gross tumor volume (GTV) is the known tumor tissue.

CTV Clinical target volume (CTV) includes GTV and volumes with subclinical disease. In this document, the CTV is defined as the GTV plus 3 mm margin. In an exemplary embodiment, this CTV is a spherical volume 30 mm in diameter.

PTV Planning target volume (PTV) encloses the CTV and outlines the margins that account for the unintended geometrical variations such that CTV is exposed to the prescribed electric field intensity with a certain probability.

TEV Total exposed volume (TEV) is defined as the tissue exposed to electric field intensities exceeding a level that is considered important for the normal tissue. In this document, this level is set as the threshold value for EP, Erev=350 V/cm.

Hot spot. In radiation therapy, hot spots are defined as regions, outside the PTV, which receive a dose larger than 100% of the prescribed PTV dose. In this document, hot spots are defined as regions in the entire TEV that are exposed to electric field intensities exceeding Eirrev=1200 V/cm.

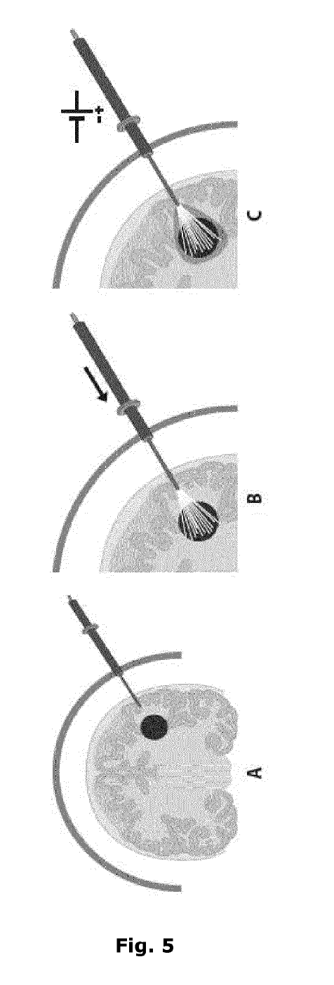

The electrode device was developed on the basis of the Electrode Introducer Device [25]—also known as “the Baseline Design”. In brief, this is a mechanical device comprising a thin shaft with individual guiding channels allowing the electrodes to move into a predefined position when deployed from a retracted position. FIG. 5 shows the treatment situation: A. The electrode device is guided to the tumor site with the aid of the stereotactic frame. B. The electrodes are deployed into the tumor. C. The electric field is delivered to the tumor. The Electrode Introducer Device facilitates the application of large field geometries using a small insertion hole, thus reducing the need for extensive surgical intervention (see FIG. 5 A).

The developmental task consisted of determining the shape, size and configuration of electrodes to obtain a device capable of delivering clinically acceptable ECT of deep-seated soft tissue tumors. The criteria of success were formulated as follows:

1. Target volume (CTV) should be exposed to electric field intensity between 350 V/cm (Erev) and 1200 V/cm (Eirrev)
2. High degree of homogeneity of the electric field intensity induced in the target volume
3. Treatable CTV size up to 3 cm diameter (spherical volume)
4. High degree of flexibility to allow targeting of different sized volumes 3 cm)
5. Minimum involvement of normal tissue
6. Easy-to-use design in clinical application

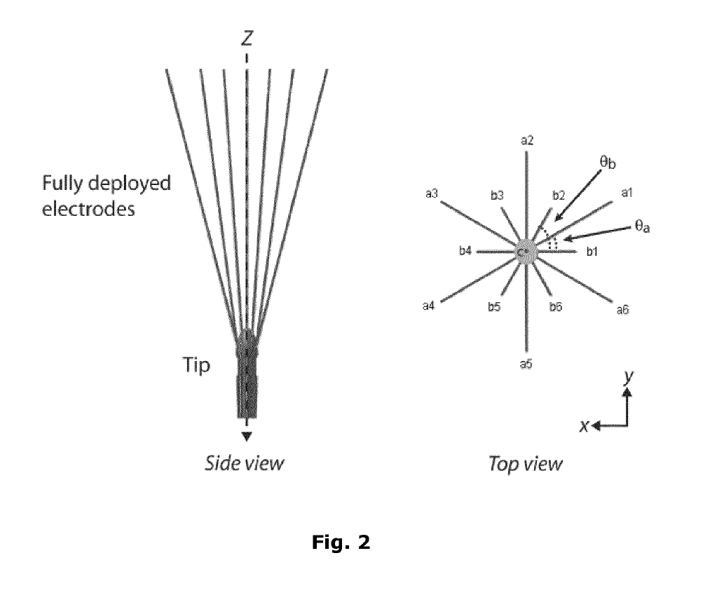

FIG. 2 shows a drawing of a preferred embodiment of the electrode device. Electrodes a1-a6 defines ring (a), electrodes b1-b6 defines ring (b), electrode c is along the z-axis which defines the axis of rotational symmetry. The position angles θa,1 and θb,2 are also shown. The device may consist of 13 cylindrical electrodes, each may be 0.30 mm in radius. 6 electrodes may be distributed evenly around the lateral surface of an imaginary cone with base radius ra and another 6 electrodes may be distributed evenly around the lateral surface of an imaginary cone with base radius rb (<ra) (see FIG. 3). FIG. 3 shows the coordinates in millimeters of the individual electrode. Left: The positions of the electrodes of ring (a), and associated position angles θa,n, n=1, . . . , 6. Right: The position of the electrodes of ring (b) and corresponding position angles θb,n, n=1, . . . , 6. The center electrode (c) is aligned with the z-axis. The two imaginary cone apices are located inside the device tip.

One electrode in the center may define the axis of symmetry. The most distal points of the electrodes may be situated in a common base plane (z=0) and may correspond to radii ra and rb, referred to as ring (a) and ring (b), respectively (see FIG. 1). As also indicated, the position angles of ring (a) and (b) are π/6 apart (see FIGS. 2 and 3). In alternative embodiments, distal points of electrodes may be situated in different planes.

The region in which the field intensity is above the threshold level Erev=350 V/cm defines the TEV.

In the treatment situation the electrode device (with retracted electrodes) may be connected to a switching or routing device that may then be connected to a pulse generator. Alternatively, the switching device may be integrated in the pulse generator. The electrode device is guided into the brain through a burr hole or after a small craniotomy using stereotactic equipment. The reference coordinates are acquired from an appropriate imaging modality like magnetic resonance imaging (MRI). When the electrode device is placed, the electrodes may gradually be deployed within the brain penetrating the tumor (see FIG. 5). After confirming the position of the device and the individual electrodes using fluoroscopy, the voltage may be applied.

The electric field E(x, y, z) in the tissue was modeled using the commercially available finite element method software, Comsol Multiphysics 3.5a (Comsol AB, Stockholm, Sweden), installed on a 64-bit Linux platform. The conductive media DC mode was applied using uniform and constant conductivities for the tissue subdomains (tumor and normal tissue). By this, it is implied that 1) the tissue is perfectly homogenous and 2) the conductivity of the tissue is field independent, i.e. the conductivity σ(x, y, z) is assumed constant during pulse delivery.

Literature suggests that the electric field distribution modeled using dynamic conductivities σ(E) encloses a slightly larger target volume for typical pulse durations and field intensities [20, 21, 26]. Due to large uncertainty related to the σ(E) relationship for brain/metastatic tissue, however, this model extension was omitted. The model therefore represents a conservative estimate of the electrode device's ability to provide tumor coverage.

Moreover, the dielectric properties of the tissue were discarded, since the polarization of the cell membrane occurs in about one microsecond [9], which is about 1/100 the duration of a typical voltage pulse in ECT, i.e. low frequency conditions apply. Although a more accurate model of the electric field is desirable in theory it has little significance for the feasibility of the presented method.

In understanding an ECT procedure, several concepts are of importance. For definition purposes, a single application of an electric field is denominated a pulse. A pulse may be delivered by a pulse generator, and pulse parameters such as amplitude, duration and number of pulses are set using the interface of the pulse generator.

A specific assigning of polarities to pairs or groups of electrodes may be denominated a polarity pattern. In traditional ECT treatments, polarity patterns were hard-wired into the generator circuitry while more advanced generators offered the user a selection between a few basic polarity patterns. Such polarity patterns would typically only be suitable for applications with the electrode devices supplied by the manufacturer of a given generator. Current state-of-the-art pulse generators comprise interfaces that allow the user limited customization options, with typical variables being numbers of active electrodes (up to seven), and applied voltages between pairs of electrodes.

Finally, a sequence of pulses—with specific pulse parameters and a specific polarity pattern—may be denominated a pulse protocol. Most ECT pulse protocols are based on a pulse sequence consisting of 8 pulses delivered at 1 pulse per second (1 Hz). Frequently, pulse protocols furthermore include the switching of polarities after each pulse application.

In addition to the novel treatment methods that have been disclosed in this document, the preferred embodiments of the treatment system including the electrode device share several novel pulse-related aspects that enhance and expand upon the capabilities of state-of-the-art pulse generators:

A first novel characteristic is the use of larger numbers of electrodes than what has previously been the norm. A preferred embodiment, as mentioned above, may use 13 electrodes in two concentric rings about a center electrode, but other embodiments have been envisioned that may apply multiple layers of electrodes to create more advanced three-dimensional geometries. In order to handle this larger number of electrodes, a preferred embodiment of the programmable switching or routing device may be configured to provide independent control of up to 32 electrodes, but larger and smaller numbers of electrodes may be also envisioned.

A second novel characteristic is the use of cone-shaped field geometries. Current state-of-the-art electrode devices share the basic concept of parallel electrodes, but the research providing the foundation for this application has shown that this concept may advantageously be abandoned. As shown in FIG. 3, the preferred embodiment of the electrode device may comprise a first cone shape with a base having a radius rₐ of 21.52 mm and a second cone shape with a base having a radius r_{b} of 10.76 mm, but other cone shapes may be envisioned.

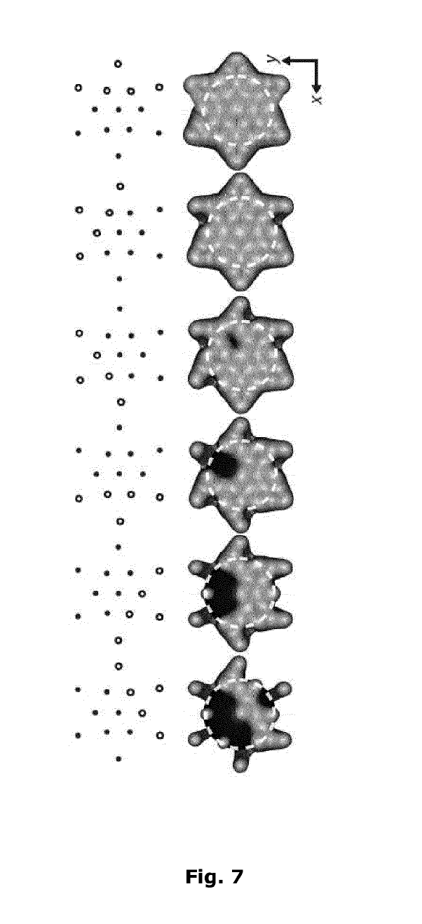

A third novel characteristic is the use of groups of electrodes with opposing polarities. Current state-of-the-art pulse protocols are based on the sequential activation of multiple pairs of electrodes, but the research providing the foundation for this application has shown that larger and more homogeneous fields may result from assigning groups of adjacent electrodes opposing polarities. FIG. 7 shows examples of employed electrode polarities. In the upper part of the figure the electrode polarities (±500 V) are illustrated with filled and open circles, respectively. The lower part of the figure shows iso-field plots at 350 V/cm. The progression may be from left to right, as each iso-field plot may be a merger of the previous plot and the immediate electric field distribution. The dashed circle indicates the position of the CTV. As shown in FIG. 7, the pulse protocol for a preferred embodiment (the “baseline design”) comprising 13 electrodes, may be based on a five-versus-eight polarity pattern.

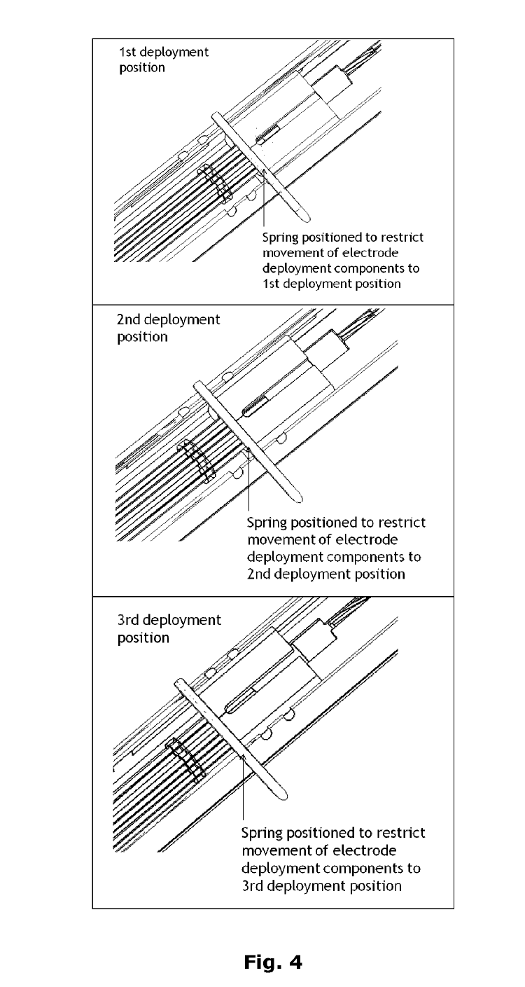

A fourth novel characteristic is the capability of the electrode device to accommodate target tissue regions of various diameters. This capability may be the result of using a cone-shaped electrode geometry and combining said geometry with the concept of flexible deployment. Flexible deployment, as mentioned above, may denote the option of retracting or advancing electrodes only partially, with either discrete or step-wise length control (see FIG. 4), and provides the physician with the option of creating a smaller field diameter by only partially deploying the electrodes. FIG. 4 shows the Flexible deployment: By placing an appropriate marker (e.g. a spring that is dimensioned to fit in a first, second and third deployment position hole in the electrode device), the physician may control the allowable deployment length of the electrodes. When combined with a cone-shaped electrode geometry as described in FIGS. 2 and 3, such control allows user-defined variations in the diameter of the field size and the resulting treatment zone (TEV) to accommodate tumors of different sizes. Of further interest, flexible deployment may be combined with variable conductive surface length and with the option of individually advancing and retracting single electrodes to create electrode devices that are truly conformal to tumor geometries. The preferred embodiment disclosed in this document provides the physician with deployment lengths 40, 46, 52 and 58 mm, corresponding to tumors in the size interval between 20 and 30 mm.

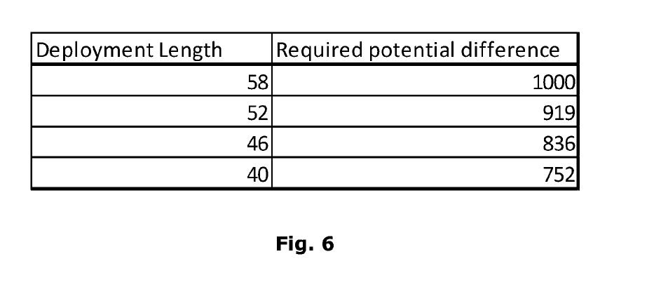

To ‘cover’ the entire CTV (3 cm diameter sphere) and using an electrode device built in accordance with the baseline design, six different polarity patterns are required (see FIG. 7). The corresponding electric field distribution is displayed as an iso-field plot, which is a visualization of the points where the electric field intensity is equal to 350 V/cm (Erev). Strictly speaking, overlapping regions receive a suboptimal pulse regime. However, the result is still valid for successful EP [27]. To obtain coverage of a CTV with a 3 cm diameter (the diameter used in the exemplary embodiment of this document), potential difference was held constant at 1000 volts. It will be obvious to those skilled in the art that CTV's—and associated tissue volumes—that are smaller may require that potential differences be smaller to maintain desired field intensities. For instance, a specific preferred embodiment of the electrode device—with deployment lengths 40, 46, 52 and 58 mm and corresponding CTV's between 20 and 30 mm—may deliver an electric field with an intensity of 350 V/cm if potential differences are reduced from 1000 V at 58 mm deployment length to 750 V at 40 mm deployment length. FIG. 6 is a table showing relations between desired deployment length and required potential differences for a specific embodiment.

Theoretically, the electrode device could be encoded with 2̂13=8192 different polarities (omitting symmetry and assuming no multiplicity from combination of different electrode polarities). The shown polarity pattern sequence (FIG. 7) was used due to largest possible margin from the 350 V/cm iso-field to the CTV edge in combination with the least number of polarity patterns required per pulse sequence (six), at 1000 V. The polarity pattern sequence was deduced from systematic testing of the physically relevant combinations using Comsol. The main characteristic of the used polarity pattern sequence can be summarized as a resemblance to a rotating parallel plate capacitor.

This particular characteristic—that may be generally applicable since the parallel plate capacitor is perceived as the optimal concept for application of an electric field to tissue—may be advantageously exploited to reduce the number of possible variants to be analyzed in e.g. the generation of a new polarity pattern.

The electrode device baseline design (FIG. 3) does not necessarily represent the optimum solution. This optimum solution may be obtained through further quantitative measures. The optimization method described here may basically be carried out by making incremental geometrical changes to the geometry of the baseline design and score each variant according to its compliance with a set of quantitative clinical criteria. The optimization may be based on a 3 cm spherical CTV.

To maintain the overall design features desirable for an electrode device for the treatment of brain cancer, the following constraints may be specified:

1. Electrode displacement is radial, with fixed apex
2. Displacement of the electrodes of ring (a) is synchronous
3. Displacement of the electrodes of ring (b) is synchronous
4. Displacements of ring (a) and (b) electrodes are independent
5. The center electrode (c) is fixed

These constraints may be applicable since rotational symmetry (relative to z-axis) applies. Displacement of individual electrodes belonging to the same ‘cone’ may not relevant since, it would imply non-symmetry.

The geometries are scored according to important clinical criteria that have been into mathematical functions, and are referred to as clinical parameters. Generally, the clinical parameters can be expressed according to any specific electropermeabilization procedure. For this case, clinical parameters that are appropriate for ECT of a tumor located within a critical organ are defined. Most of the clinical parameters shown below may be formulated using ideas from radiation therapy and not necessarily representative for other paradigms, however, the concept applies to other formulations analogously.

Tumor Control.

One of the main objectives of cancer treatment in general may be to treat sufficiently to prevent proliferation and regrowth. Tumor response levels may be facilitated through good agreement between prescribed and delivered field intensity. A parameter L(Vlow) that takes into account the influence of Vlow, the size of the region within the CTV that is exposed to an electric field intensity lower than a the empirically determined threshold value of Erev=350 V/cm, may be defined:

$$L ( {V}_{low} ) = \{ \begin{matrix}0 & for {V}_{low} > 0 \\ 1 & for {V}_{low} = 0\end{matrix}$$

L(Vlow) indicates a null-tolerance enforcement, i.e. if any fraction of the CTV is undertreated it will lead to a rejection of the particular design (score=0).

Hot Spots.

At high electric field intensities (E>Eirrev) the cells within the exposed tissue die. This may be an unwanted outcome if the electrode device is applied in gene electrotransfer where the viability of cells is crucial, or if the electrodes responsible for creating the hot spots are placed in healthy and/or critical structures. H(Vhigh) is a parameter that takes into account the extent of Vhigh, the amount of tissue exposed to electric field intensities higher than Eirrev=1200 V/cm:

$$H ( {V}_{high} ) = 1 - \frac{{V}_{high}}{CTV}$$

Average Electric Field.

The uncertainties associated with the calculation of electric field, implying that the chance of staying inside the limits of reversible EP may be improved, if the mean electric field intensity is close to the interval median (775 V/cm). M(μ) account for the deviation of the mean of the induced electric field intensity μ from the interval median:

$$M ( μ ) = \{ \begin{matrix}1 - \frac{\sqrt{{( μ - 775 )}^{2}}}{425} & for 350 < μ < 1200 \\ 0 & all other values of μ\end{matrix}$$

Electric Field Homogeneity.

S(λ) is a parameter that takes into account the influence of λ, the average distance from the mean electric field intensity within CTV (log-transformed data). It is defined for λ>0:

S(λ)=1/λ

S(λ) is normalized to 1.

Normal Tissue Sparing.

Finally, the amount of normal tissue exposed to an electric field intensity above Erev and Eirrev, respectively needs to be addressed. Normal tissue may be defined as the total exposed volume (TEV) minus the clinical target volume (CTV) (see FIG. 1).

Involvement of normal tissue is only regarded when two or more otherwise equally good device geometries are matched. In such cases, the geometry including the least amount of normal tissue may be preferred. However, it is important to realize that it is not possible to state a generalized expression concerning normal tissue because of the complexity of the brain. In other words, it is not guaranteed that an overall reduction of the normal tissue involvement decreases the exposure of the most critical regions within the normal tissue.

Collecting all clinical parameters L,H,M,S into one expression yields the objective function:

A(L,H,M,S)_{Δa,Δb}=L(V_{low})H(V_{high})M(μ)S(λ)

where Δ and Δb indicate the geometrical change of ring (a) and (b) electrodes, respectively, measured as radial change of the electrode ends in units of millimeter (mm). For instance, the baseline design corresponds to Δa=0,Δb=0 (FIG. 3).

Negative values of Δa and Δb correspond to inward displacements (reduced radius of ring (a) and (b)), and positive values mean outward displacements (increased radius of ring (a) and (b)). Each clinical parameter yields a value between 0 and 1. Consequently, A(L,H,M, S) yields a value between 0 and 1, which is called the ‘score’. A high score is associated with good compliance with the clinical parameters, accordingly, the optimum geometry is found by maximizing the objective function:

$${max}_{{Δ}_{a} , {Δ}_{b}} {A ( L , H , M , S )}_{{Δ}_{a} , {Δ}_{b}}$$

The objective function was calculated using the scripting environment Matlab R2006a (The Mathworks, Inc., MA, USA) in combination with Comsol. The calculation was based on electric field data of 16,333 sampling points in the defined CTV. The points were distributed as vertex points of a regular tetrahedral grid, corresponding to a voxel size of 0.87 mm3. Using a tetrahedral grid instead of e.g. mesh node points generated in the finite element calculation or ordinary Cartesian grid points, advantage was taken of the invariance of this grid to the π/3 rotations of the electrode polarities during a pulse (FIG. 7). This allowed the imposing of rules concerning the overlapping regions, for example that any given grid point was assigned with the highest electric field intensity induced at that point during the course of a pulse (six electrode polarities).

In general, geometrical errors follow the Gaussian distribution with a mean and a standard deviation. Errors are typically divided into systematic errors and random errors. Systematic errors arise in the preparation stage of a treatment and include setup errors, target volume delineation errors, equipment calibration errors, calculation errors and organ motion in imaging scanner. Such errors are especially critical in the treatment of brain cancers, where errors may lead to the inadvertent loss of critical functions. The electrode device described in this document offers the physician the option of interrogating tissue that is to be treated with the purpose of determining whether patient health may be adversely impacted by the treatment itself. Such interrogation, as described in this document, may consist of applying to a given pair of electrodes that are placed in a given tissue region a test current sufficient to cause transient suppression of normal tissue functions. Provided that the patient undergoing treatment is awake, it will be possible to determine the impact of the test current application treatment, which will enable the physician to make changes to the treatment plan to avoid critical structures.

Random error includes target motion, unforeseen tissue variations, inaccuracies resulting from the deflection of electrodes from their planned trajectories as well as variations in patient setup and equipment if treatment is fractionized. Although errors can be reduced with correction procedures, e.g. setup verification imaging, corrections typically do not correct organ motion, calculation errors and target volume delineation inaccuracies. Furthermore, because of the limited accuracy of the correction procedures, they introduce errors as well. To account for the residual error, geometrical margins are always required and should be used in the treatment plan according to a margin recipe that predicts the level of tumor control in a predefined percentage of the patients [28, 29].

With ECT using the electrode device, it is necessary to address two independent geometrical margins, one is related to the inaccuracy in positioning the electrode device entity, called “CTV margin”, and the other is related to the displacement of the electrodes during deployment in the tissue, called “deflection margin”. (CTV margin is the expansion of the CTV to yield the PTV). It is not possible to choose these margins since the uncertainties involved in the treatment procedure are not quantified yet. Neither are the tumor control probabilities for different electric field intensities. Instead the “CTV tolerance” and the “deflection tolerance”, which are the corresponding device related geometrical tolerances, are reported. The CTV tolerance is defined as the difference between the 30 mm spherical CTV to the maximum spherical volume that is covered by the 350 V/cm iso-field, and the deflection tolerance as the maximum deflection of the electrodes of ring (a) and (b), respectively, maintaining the coverage of the 30 mm spherical CTV. The optimization process described in section 2.4 provides data to assess the deflection and CTV tolerances. Ideally, the CTV tolerance and CTV margin should be equal, to achieve the expected tumor control probability and secure normal tissue sparing. In contrast, the deflection tolerance should as minimum be the size the deflection margin since deflection tolerance primarily is an indicator of the geometrical robustness which should be as great as possible. In the present ECT procedure corrections based on on-line fluoroscopy and implanted markers will be implemented to reduce margins.

To distinguish between the intentional electrode displacements introduced in the optimization section (2.4) and the unintentional electrode displacement in the treatment situation, the latter is denoted electrode deflection.

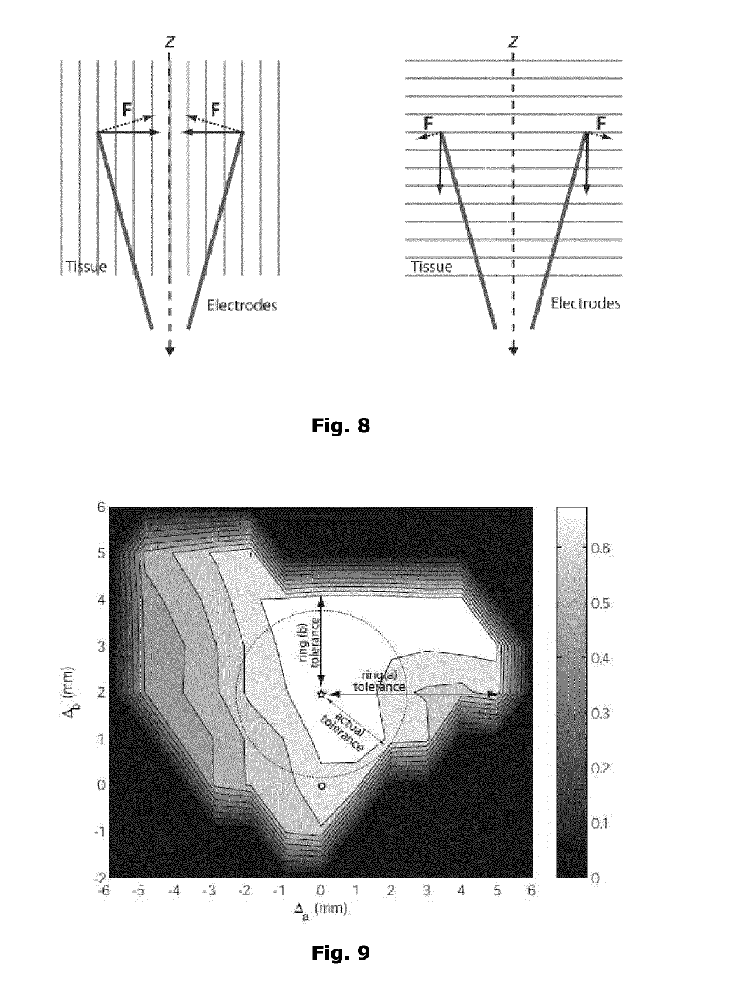

The electrode deflection can be understood if anisotropic target tissue (e.g. tumors) is considered as represented by two orthogonal linear structures. The interaction between the structures and the electrodes is illustrated in FIG. 8. FIG. 8 shows an example of how the electrodes can deflect inwards or outwards depending on the anisotropy. Two orthogonal linear anisotropies illustrate the possible outcome under symmetry conditions. The resultant force F indicates the direction of electrode deflection, the relative size of the vectors does not necessarily correspond to the actual situation. In one case the electrodes experience an inward force and in the other situation the force is directed outward, depending on the anisotropy, causing a reduction and increase of the electrode ring radii, respectively. In both cases the deployment direction of the electrodes changes. Obviously, perfectly homogenous tissue will only exert a force exactly opposing the movement of the electrodes with no lateral force components and no deflection would be expected.

In reality any electrode will deform if the resistance in its path is high enough. The electrodes used in the manufacture of the electrode device are made of a biocompatible alloy with high strength and corrosion resistance. Preliminary tests have been carried out to monitor the directional stability of the electrodes during the deployment procedure using various animal tissues. Although this turned out satisfactory further testing is needed to specify the definite operational uncertainty (and deflection margin).

The positioning error in this case is mainly resulting from the mechanical inaccuracy of the stereotactic frame and MRI/fluoroscopy errors due to limited resolution and artifacts. Target volume delineation does not introduce a large error since brain metastases are usually very well-defined on MRI scans. Patient movements during treatment is also regarded a minor source of error since the electrode device is fixed to the skull by the stereotactic frame. Calculation error should be considered a relatively large uncertainty.

The CTV tolerance is found by incremental enlargement of the spherical volume

(using Matlab scripting with Comsol) to find the limit of the coverage.

FIG. 9 shows the objective function scores in grey scale. The small circle at (Δa=0, Δb=0) indicates the position of the baseline and the star at (Δa=0, Δb=2) shows one of the improved geometries. The large dashed circle shows the actual deflection tolerance in comparison to the independent deflection tolerances of ring (a) and (b) electrodes. Neighboring geometries are connected using linear interpolation. The objective function A(L,H,M, S) may be calculated for all combinations of the displacements Δa={−6, −5, −4, −3, −2, −1, 0, 1, 2, 3, 4, 5, 6} and Δb={−2, −1, 0, 1, 2, 3, 4, 5, 6} and the scores are shown in FIG. 9 as a 2D score map. In particular (Δa=0,Δb=0) means zero displacement which corresponds to the electrode device baseline design. Negative values of Δa and Δb, respectively, are equivalent to an inward displacement of the electrodes and positive values are equivalent to an outward displacement of the electrodes relative to the baseline, measured in millimeter (mm) at the electrode ends. A quick visual inspection identifies several combinations of Δa and Δb as high scoring geometries (FIG. 9). In table 1 the clinical parameters of the objective function are specified for the top 5 scoring device geometries and compared to the baseline design. Maximum score is 0.74 found at (Δa=1, Δb=2) and (Δa=0, Δb=3), suggesting a displacement of the electrodes of ring (a) by 1 mm and 0 mm outwards, respectively, and displacement of the electrodes of ring (b) by 2 mm and 3 mm outwards, respectively. The amount of involved normal tissue is practically equal for the geometries (Δa=1, Δb=2) and (Δa=0, Δb=3).

Table 1 shows deflection tolerances of ring (a) and (b) of the top 5 and baseline geometries. E.g. for geometry (Δa=1, Δb=2) a 4 mm inward or outward deflection of ring (a) electrodes is tolerated, and only a 2 mm inward or outward deflection of ring (b) electrodes is tolerated. In other words the electrodes of ring (a) can travel up to 4 mm and still cover the CTV sufficiently, whereas the ring (b) electrodes can travel only 2 mm. The deflection tolerances are thus a measure of the geometrical robustness of a particular device geometry. Equivalently, using the score map in FIG. 9, one can read off the distance from the point representing a particular geometry to the beginning of the area with quickly diminishing score. For example, for geometry (Δa=0, Δb=2) the ring (b) electrodes have to travel a small distance (2 mm) to reach the “precipice” whereas the same device geometry has much larger tolerance (5 mm) of the ring (a) electrode deflection.

The tabulated data (table 1) also show the tolerance of the positioning of the electrode device (CTV tolerance). E.g. the largest spherical CTV that can be covered by the 350 V/cm iso-field expanded by the (Δa=0, Δb=2) geometry has a radius of 15.5 mm, i.e. 0.5 mm larger than the CTV used in the optimization process. This implies a 0.5 mm CTV tolerance of this geometry, meaning that the PTV has 0.5 mm margin to the CTV. The data also shows that the CTV tolerance is independent of the device geometry since the tolerance levels are the same for all geometries.

The scores and the normal tissue involvement of the top 5 device geometries are not very far apart, however, their individual robustness is quite different. This suggests that in practice the device geometry with the largest deflection tolerance should be selected as the optimum device geometry.

The device geometry (Δa=0, Δb=2) qualifies as the most robust geometry. The generalized deflection tolerance is given by the smallest tolerance of ring (a) and (b), i.e. 2 mm. This is an approximation since the deflection tolerances of ring (a) and ring (b) are not completely independent, and the actual generalized deflection tolerance might be slightly smaller. However, if the deflections are treated separately, ring (a) exhibit 5 mm tolerance, both inwards and outwards, and ring (b) exhibit 2 mm tolerance in both directions (FIG. 9).

TABLE 1
The top 5 scoring electrode geometries and the prototype geometry in the
bottom. ‘tolerances’ indicate the allowed deflections of the electrodes
and the allowed positional error of the device. ‘normal tissue’ gives
calculated fractions of the normal tissue in the TEV: ‘rev’ is the part
exposed to electric field intensities from Eᵣₑᵥ to Eᵢᵣᵣₑᵥ, and
‘irrev’ is the part exposed to electric field intensities above Eᵢᵣᵣₑᵥ.

| | | | | | | | tolerances | | | |
|---|---|---|---|---|---|---|---|---|---|---|
| | | | | | | | (mm) | | | |
| | | | | | | | deflec- | | normal | |
| | | | | | | | tion | | tissue (%) | |
| Δₐ | Δ_{b} | L | H | M | S | score | a, b | CTV | rev | irrev |
| 1 | 2 | 1 | 0.95 | 0.96 | 0.81 | 0.74 | 4, 2 | 0.5 | 56.0 | 4.4 |
| 0 | 3 | 1 | 0.95 | 0.96 | 0.81 | 0.74 | 5, 1 | 0.5 | 55.9 | 4.6 |
| 0 | 4 | 1 | 0.95 | 0.92 | 0.84 | 0.73 | 4, 0 | 0.5 | 56.5 | 4.6 |
| 0 | 2 | 1 | 0.94 | 0.98 | 0.79 | 0.73 | 5, 2 | 0.5 | 55.2 | 4.5 |
| 2 | 4 | 1 | 0.96 | 0.84 | 0.87 | 0.70 | 2, 0 | 0.5 | 58.5 | 4.2 |
| 0 | 0 | 1 | 0.93 | 0.96 | 0.73 | 0.65 | 1, 1 | 0.5 | 53.2 | 4.5 |

### Discussion

A simple way of incorporating a set of clinical parameters into a specific design optimization task has been demonstrated. The method was applied to a novel electrode device invented to allow electropermeabilization of deep seated tumors, primarily intracranial metastases, with the purpose of establishing an optimal device geometry. Further applications of the device include gene electrotransfer and irreversible electroporation, which have been addressed only briefly in this paper. It has also been shown that applications of the same optimization methodology may enable a physician to rapidly adapt a treatment plan to changing circumstances.

A secondary purpose of this work was to draw attention to the importance of quantitative approaches in clinical EP since it is a fast emerging technology and should be exploited in the most advantageous way. Such a goal is only achievable if different strategies are tested, and since ECT faces challenges similar to those in radiation therapy in terms of tumor coverage and normal tissue sparing, some of the same ideas and terminology have been adopted.

A number of assumptions and simplifications have been made to demonstrate the feasibility of the presented optimization. E.g. a correct electric field model would have included at least the non-linear dynamics due to the σ(E) relationship [30]. On the other hand, no usable data to implement the dynamics of the conductivity into the model was available. An improvement would be to use slightly different threshold levels to observe the sensitivity of the method. However, this was not considered important for demonstrating the general feasibility of the method. The macroscopic heterogeneity of the target tissue should also be determined to make realistic models since it is often an indication of heterogeneous conductivity of the target tissue [31] which affects the local electric field. Due to shortage of important electrical data of the tissue, a simple model which presumably slightly underestimates the electric field intensities [20, 21, 26] was preferred.

In the present work the EP threshold levels Erev and Eirrev for brain metastases were extracted from results of preclinical studies and reported values from other tissues, since specific data was not found in the literature. Improvements to the mathematical expressions L, H, M, S of the clinical parameters demand better understanding of tissue reaction and higher accuracy of threshold levels.

Other optimization methods are possible, e.g. genetic algorithms [32, 33] or Theil's inequality Coefficient [17, 34]. However, genetic algorithm requires much calculation time when more than a few parameters are used and Theil's inequality is very sensitive to additive transformation [34], which is important when for example the EP thresholds are quite uncertain. Another advantage of the method proposed in this document is the ease with which one may see the value of the individual clinical parameter and decide which geometry is optimal in each case. For example, normal tissue involvement sometimes constitutes a serious matter and other times it is of less concern if the involved region coincides with a non-critical structure or necrosis due to previous treatments. In radiation therapy the conformity index is sometimes applied as a tool for attributing a score to a treatment plan and comparing several treatment plans for the same patient, very similar to the scoring system presented in this document [35]. The much more commonly used dose-volume histograms provide statistical information about absorbed dose distribution in the treated tissue and do not provide a good indication of conformity. However, similar histograms could serve as a complementary tool in ECT if the absorbed dose is substituted with for example the electric field intensity.

The results showed that several alternatives to the baseline comply better with the treatment criteria and also show greater robustness in terms of deflection tolerance of the electrodes, and led to appropriate modifications.

The optimization data show small differences in the overall scores, even between quite different device geometries. Involvement of normal tissue is also quite comparable between geometries. Most critical may be the fraction of irreversibly electropermeabilized tissue that amounts to about 4.5% only. This number is of course uncertain because it is dependent on the accuracy of the threshold level for irreversible EP. The largest differences are seen in the deflection tolerances (table 1). It is premature to state which level of deflection tolerance is required since the deflection margin are yet to be assessed from clinical trials and other tests, but since the scores are very similar the device geometry with the largest deflection tolerance should be selected. As far as the CTV tolerance is concerned it seems unrealistic that 0.5 mm will suffice. The planned correction procedures (e.g. fluoroscopy) reduce the uncertainty of the stereotactic frame, which normally can be considered to be 1-2 mm. Furthermore, the uncertainty of the imaging process and morphological/translational changes of the GTV due to the delay between the time of imaging and the time of treatment (which can amount to days) is also reduced by the online correction procedure. However, the target volume delineation error, the calculation error and the residual error will remain, demanding a certain CTV margin. A realistic guess of the CTV margin would be about 1-2 mm when on-line correction procedures are implemented and about the double (4-5 mm) when no on-line correction procedures are used. This suggests that the maximum treatable volume (CTV) should be reduced from 30 mm to around 26-28 mm in diameter (with correction procedures).

In the exemplary embodiment disclosed in this document, only the maximum target volume (30 mm in diameter) has been addressed. In light of the advances towards treatment planning of ECT [18] it may be be required that the electrode device is adjustable so that different volumes can be targeted without having to involve a larger fraction of normal tissue. As mentioned above, the electrode device presented in this paper has a feature, referred to as “flexible deployment”, to accommodate the variation in the metastasis sizes. Flexible deployment allows the electrodes to be deployed in different lengths meaning that the corresponding imaginary cones will have different sizes, but same proportion between base plane and height. This means that, to a good approximation, the electric field distribution is accordingly scaled if the voltage is reduced by the reciprocal value of the fraction of the cone sizes. Optimization of other CTV diameters will therefore yield the same scores and relative geometrical tolerances as for the maximum CTV. A particular embodiment of the electrode device has.

### CONCLUSION

In this document, the ICRU formalism from radiation oncology has been adopted to optimize the electrode device for intracranial EP by assessing different device geometries in terms of tumor control, hot spots, average electric field intensity, electric field distribution and normal tissue involvement. Based on predefined levels of EP thresholds and objective function formulation, better performing alternatives to the baseline design have been successfully obtained. Geometrical uncertainties related to the EP treatment, using the electrode device, have also been addressed. In conclusion, the geometrical robustness (deflection tolerance) of the electrode device is improved with small adjustments of the baseline design. The positioning tolerance (CTV tolerance) of the device indicates that the largest treatable CTV must be reduced from 30 mm in diameter to around 26-28 mm in diameter to ensure coverage, given that on-line correction procedures are applied.

In spite of the fact that the electric field model employed in this work is lacking key features such as dynamic conductivity and structural heterogeneity of the tissue, the overall feasibility of the method has been demonstrated and it has been shown that the application of this method may have important advantages in the adaptation of a treatment plan to changing circumstances.

The construction of the objective function was based on a mix of well known principles of radiation oncology and empirical data of EP. Further studies may improve biological verification.

### Incorporation of Previous Documents

US 2009/0254019 is hereby incorporated by reference in its entirety in this application.

### REFERENCES

[1] E. Neumann, M. Schaeferridder, Y. Wand, P. H. Hofschneider, Gene transfer into mouse lyoma cell
   by electroporation in high electric fields, EMBO J 1 (7) (1982) 841-845.
[2] A. I. Daud, R. C. DeConti, S. Andrews, P. Urbas, A. I. Riker, V. K. Sondak, P. N. Munster, D. M.
Sullivan, K. E. Ugen, J. L. Messina, R. Heller, Phase i trial of interleukin-12 plasmid electroporation
   in patients with metastatic melanoma, J Clin Oncol 26 (36) (2008) 5896-5903.
[3] D. Ferber, Safer and virus-free?, Science 294 (5547) (2001) 1638-1642.
[4] S. Orlowski, J. Belehradek Jr, C. Paoletti, L. M. Mir, Transient electropermeabilization of cells
   in culture: Increase of the cytotoxicity of anticancer drugs, Biochem Pharmacol 37 (24) (1988) 4727-4733.
[5] J. Belehradek Jr, S. Orlowski, B. Poddevin, C. Paoletti, L. M. Mir, Electrochemotherapy of spontaneous mammary tumours in mice, Eur J Cancer Clin Oncol 27 (1) (1991) 73-76.
[6] M. Marty, G. Sersa, J. R. Garbay, J. Gehl, C. G. Collins, M. Snoj, V. Billard, P. F. Geertsen,
J. O. Larkin, D. Miklavcic, I. Pavlovic, S. M. Paulin-Kosir, M. Cemazar, N. Morsli, D. M. Soden,
Z. Rudolf, C. Robert, G. C. O'Sullivan, L. M. Mir, Electrochemotherapy—an easy, highly effective and safe treatment of cutaneous and subcutaneous metastases: Results of ESOPE (european standard operating procedures of electrochemotherapy) study, EJC Supplements 4 (11) (2006) 3-13.
[7] P. Hojman, H. Gissel, F. M. Andre, C. Cournil-Henrionnet, J. Eriksen, J. Gehl, L. M. Mir, Physiological effects of high- and low-voltage pulse combinations for gene electrotransfer in muscle, Hum Gene Ther 19 (11) (2008) 1249-1260.
[8] G. Saulis, G. Aulis, M. S. Venslauskas, J. Naktinis, Kinetics of pore resealing in cell-membranes after electroporation, Bioelectrochem Bioenerg 26 (1) (1991) 1-13.
[9] M. P. Rols, J. Teissie, Electropermeabilization of mammalian cells. quantitative analysis of the phenomenon, Biophys J 58 (5) (1990) 1089-1098.
[10] R. Heller, R. Gilbert, M. J. Jaroszeski, Clinical applications of electrochemotherapy, Adv Drug Deliv Rev 35 (1) (1999) 119-129.
[11] M. Marty, G. Sersa, J. R. Garbay, J. Gehl, C. G. Collins, M. Snoj, V. Billard, P. F. Geertsen,
J. O. Larkin, D. Miklavcic, I. Pavlovic, S. M. Paulin-Kosir, M. Cemazar, N. Morsli, D. M. Soden,
Z. Rudolf, C. Robert, G. C. O'Sullivan, L. M. Mir, Electrochemotherapy—an easy, highly effective and safe treatment of cutaneous and subcutaneous metastases: Results of ESOPE (european standard operating procedures of electrochemotherapy) study, EJC Supplements 4 (11) (2006) 3-13.
[12] L. M. Mir, J. Gehl, G. Sersa, C. G. Collins, J.-R. Garbay, V. Billard, P. F. Geertsen, Z. Rudolf, G. C.
O'Sullivan, M. Marty, Standard operating procedures of the electrochemotherapy: Instructions for the use of bleomycin or cisplatin administered either systemically or locally and electric pulses delivered by the cliniporator (TM) by means of invasive or non-invasive electrodes, EJC Supplements 4 (11) (2006) 14-25.
[13] L. G. Campana, S. Mocellin, M. Basso, O. Puccetti, G. L. D. Salvo, V. Chiarion-Sileni, A. Vecchiato,
L. Corti, C. R. Rossi, D. Nitti, Bleomycin-based electrochemotherapy: Clinical outcome from a single institution's experience with 52 patients, Ann Surg Oncol 16 (1) (2009) 191-199.
[14] L. W. Matthiessen, C. Kamby, H. W. Hendel, H. H. Johannesen, J. Gehl, Electrochemotherapy as palliative treatment for chest wall recurrence of breast cancer—initial results, EJC Supplements 7 (2) (2009) 273-273.
[15] D. Miklavcic, K. Berays, D. Semrov, M. Cemazar, F. Demsar, G. Sersa, The importance of electric field distribution for effective in vivo electroporation of tissues, Biophys J 74 (5)
[16] D. Miklavcic, S. Corovic, G. Pucihar, N. Payselj, Importance of tumour coverage by sufficiently high local electric field for effective electrochemotherapy, EJC Supplements 4 (11) (2006) 45-51.
[17] D. Sel, A. M. Lebar, D. Miklavcic, Feasibility of employing model-based optimization of pulse amplitude and electrode distance for effective tumor electropermeabilization, IEEE Trans Biomed Eng 54 (5) (2007) 773-781.
[18] D. Miklavcic, M. Snoj, A. Zupanic, B. Kos, M. Cemazar, M. Kropivnik, M. Bracko, T. Pecnik, E. Gadzijev, G. Sersa, Towards treatment planning and treatment of deep-seated solid tumors by electrochemotherapy, Biomed Eng Online 9 (10).
[19] U. Pliquett, R. Langer, J. C. Weaver, Changes in the passive electrical properties of human stratum corneum due to electroporation, Biochim Biophys Acta 1239 (2) (1995) 111-121.
[20] D. Sel, D. Cukjati, D. Batiuskaite, T. Slivnik, L. M. Mir, D. Miklavcic, Sequential finite element model of tissue electropermeabilization, IEEE Trans Biomed Eng 52 (5) (2005) 816-827.
[21] N. Payselj, Z. Bregar, D. Cukjati, D. Batiuskaite, L. M. Mir, D. Miklavcic, The course of tissue permeabilization studied on a mathematical model of a subcutaneous tumor in small animals, IEEE Trans Biomed Eng 52 (8) (2005) 1373-1381.
[22] B. Agerholm-Larsen, H. Iversen, P. Ibsen, J. Moeller, L. Staal, L. G. Salford, J. Gehl, Electrochemotherapy for brain tumours investigated in vivo, Neuro Oncol 11 (5) (2009) 597-597.
[23] J. Gehl, T. H. Sørensen, K. Nielsen, P. Raskmark, S. L. Nielsen, T. Skovsgaard, L. M. Mir, In vivo electroporation of skeletal muscle: Threshold, efficacy and relation to electric field distribution, Biochim Biophys Acta 1428 (2-3) (1999) 233-240.
[24] International Commission on Radiation Units and Measurements, Prescribing, recording, and reporting photon beam therapy, Tech. Rep. ICRU report 50, ICRU (1993).
[25] J. Gehl, K. Videbaek, patent no. WO/2007/144004.
[26] N. Payselj, D. Miklavcic, Numerical models of skin electropermeabilization taking into account conductivity changes and the presence of local transport regions, IEEE Trans Plasma Sci 36 (4) (2008) 1650-1658.
[27] P. J. Canatella, J. F. Karr, J. A. Petros, M. R. Prausnitz, Quantitative study of electroporation-mediated molecular uptake and cell viability, Biophys J 80 (2) (2001) 755-764.
[28] A. Dutreix, When and how can we improve precision in radiotherapy?, Radiother Oncol 2 (1984) 275-292.
[29] M. V. Herk, P. Remeijer, J. V. Lebesque, Inclusion of geometric uncertainties in treatment plan evaluation, Int 3 Radiat Oncol Biol Phys 52 (5) (2002) 1407-1422.
[30] N. Payselj, D. Miklavcic, Irreversible Electroporation, Springer-Verlag Berlin Heidelberg, 2010.
[31] E. Gersing, Impedance spectroscopy on living tissue for determination of the state of organs, Bio-electrochem Bioenerg 45 (2) (1998) 145-149.
[32] D. S. Weile, E. Michielssen, Genetic algorithm optimization applied to electromagnetics: A review, IEEE Trans Antennas Propag 45 (3) (1997) 343-353.
[33] S. Corovic, A. Zupanic, D. Miklavcic, Numerical modeling and optimization of electric field distribution in subcutaneous tumor treated with electrochemotherapy using needle electrodes, IEEE Trans Plasma Sci 36 (4) (2008) 1665-1672.
[34] R. M. Leuthold, Use of theil's inequality coefficients, Am J Agric Econ 57 (2) (1975) 344-346.
[35] L. Feuvret, G. Noel, 3.3. Mazeron, P. Bey, Conformity index: A review, Int 3 Radiat Oncol Biol Phys 64 (2) (2006) 333-342.

1. A method for treating a patient by covering a tissue region with an electric field capable of transiently or permanently permeabilizing cells in said tissue region, comprising the steps of:
a. positioning the electrodes of an electrode device in a tissue region to be treated; and
b. creating a specific polarity pattern by applying to a first subset of electrodes comprising at least two electrodes a first polarity while at the same time applying to a second subset of electrodes comprising at least two electrodes a second polarity.

2. A method according to claim 1, said first subset of electrodes comprising five electrodes and said second subset of electrodes comprising eight electrodes.

3. A method according to claim 2, said electrodes are arranged relative to a polar coordinate system such that:
said electrodes in said first subset are arranged at the angular positions 210, 240, 270, 300 and 330 degrees in the said polar coordinate system,
said electrodes in said second subset are arranged at the angular positions 0, 30, 60, 90, 120, 150 and 180 degrees in said polar coordinate system, and
a center-electrode is arranged in the center of said polar coordinate system such that said center-electrode defines a normal to said polar coordinate system.

4. A method according to claim 3, further comprising the step of:
c. repeating step b. at least once.

5. A method according to claim 4, further comprising the step of:
d. repeating step c. while rotating the polarity pattern either 60, 120, 180, 240 or 300 degrees relative to the center of the said polar coordinate system.

6. A method according to claim 3, further comprising the step of:
c. repeating step b. 5 times in such a way that the polarity pattern has been rotated 60, 120, 180, 240 and 300 degrees relative to the center of the said polar coordinate system.

7. A method according to claim 6, further comprising the step of:
d. repeating step c. at least once.

8. A method according to claim 3, further comprising the step of:
c. reversing said application of polarities at least once.

9. A method according to claim 8, further comprising the step of:
d. repeating steps b. to c. while rotating the polarity pattern either 60, 120, 180, 240 or 300 degrees relative to said polar coordinate system.

10. A method according to claim 8, further comprising the step of:
d. repeating step b. to c. while sequentially rotating the polarity pattern either 60, 120, 180, 240 and 300 degrees between each repetition relative to said polar coordinate system.

11. A method according to claim 8, further comprising the steps of:
d. rotating the polarity pattern either 60, 120, 180, 240 or 300 degrees relative to said polar coordinate system, and
e. repeating steps c. to d. in any order so that at least one polarity reversal has been applied to at least one polarity pattern.

12. A method according to claim 8, further comprising the steps of:
d. rotating the polarity pattern either 60, 120, 180, 240 or 300 degrees relative to said polar coordinate system, and
e. repeating steps c. to d. in any order so that at least one polarity reversal has been applied to each polarity pattern.

13. A method according to claim 3, further comprising the steps of:
c. reversing said application of polarities,
d. rotating the polarity pattern either 60, 120, 180, 240 or 300 degrees relative to said polar coordinate system, and
e. repeating steps c. to d. in any order so that at least one polarity reversal has been applied to at least one polarity pattern and that all the polarity patterns have had an equal number of polarity reversals applied.

14. A method according to claim 3, further comprising the steps of:
c. reversing said application of polarities,
d. rotating the polarity pattern either 60, 120, 180, 240 or 300 degrees relative to said polar coordinate system, and
e. repeating steps c. to d. in any order so that at least one polarity reversal has been applied to each polarity pattern and that all the polarity patterns have had an equal number of polarity reversals applied.

15. A treatment system for the treatment of cancers and other diseases, comprising:
a pulse generating device;
a switching device; and
an electrode device with a symmetry axis, wherein the devices are in operative connection, the electrode device comprises at least two electrodes for placement on a tissue region to be treated and for creation of a specific polarity pattern, wherein the switching device is adapted to assign a specific polarity to each of the electrodes, wherein the switching device is adapted to activate and deactivate the electrodes so as to define a treatment volume of variable dimensions and geometry.

16. A treatment system in accordance with claim 15, wherein the electrode device comprises at least 8 electrodes.

17. A treatment system in accordance with claim 15, wherein the electrode device comprises at least 13 electrodes.

18. A treatment system in accordance with claim 15, where a first electrode C is co-aligned with the symmetry axis of the electrode device.

19. A treatment system in accordance with claim 18, where said first electrode C is defined as the origo in a coordinate system, when said electrode device is viewed along said symmetry axis.

20. A treatment system in accordance with claim 15, where at least some of said electrodes are non-parallel.

21. A treatment system in accordance with claim 15, wherein each electrode defines a distal tip and wherein the electrodes define a first and a second group of electrodes, wherein the distal tip of each of the electrodes in the first group defines a first circle having a center which coincides with the axis of symmetry; and wherein the distal tip of each of the electrodes in the second group defines a second circle which has a center which coincides with the axis of symmetry, and wherein the axis of symmetry defines a normal to the plane defines by each of the first and the second circle.

22. A treatment system according to claim 21, wherein the electrodes of the first group defines a first cone (cone A) by extending along the sides of the first cone and such that the distal tip of the electrodes terminates at the base of the first cone, and wherein the electrodes of the second group defines a second cone (cone B) by extending along the sides of the second cone and such that the distal tip of the electrodes terminates at the base of the first cone.

23. A treatment system according to claim 21, wherein the first circle is concentric with the second circle.

24. A treatment system according to claim 21, wherein the first and second circles are concentric, having centers coinciding with an electrode C that is co-aligned with the symmetry axis of the electrode device.

25. A treatment system according to claim 21, wherein the radius of the first circle is larger than the radius of the second circle.

26. A treatment system in accordance with claim 21, wherein the electrodes of the first group are equidistantly distributed along the circumference of the first circle, and wherein the electrodes of the second group are equidistantly distributed along the circumference of the second circle.

27. A treatment system in accordance with claim 15, wherein the first group of electrodes comprises 6 electrodes, and wherein the second group of electrodes comprises 6 electrodes.

28. A treatment system in accordance with claim 15, wherein the electrodes of the first group are distributed along a first circle such that the electrodes are provided at the positions 0, 60, 120, 180, 240, 300 degrees in a polar coordinate system which is provided in the plane of the first circle and which has its center at the center of the first circle, and wherein the electrodes of the second group are distributed along a second circle such that the electrodes are provided at the positions 30, 90, 150, 210, 270 and 330 degrees in a polar coordinate system which is provided in the plane of the first circle and which has its center at the center of the first circle.

29. A method for treating a patient according to claim 1, further comprising the step of mapping the tissue region to be treated before initiating the positioning and creating.

30. A method in accordance with claim 29, wherein mapping is done by applying to pairs of electrodes a test current and monitoring tissue or patient response to said test current application.

31. A method in accordance with claim 29, wherein a particular tissue area within the tissue region to be treated is excluded from treatment based on the results of the mapping.

32. A method in accordance with claim 31, wherein exclusion of treatment of the particular tissue area within the tissue region treated is done by deactivating those electrodes placed in the particular tissue area, resulting in the creation a new treatment volume.

33. A method in accordance with claim 32, where the continued capability of the electric field to create transient or permanent permeabilization of the tissue in the new treatment volume is caused by a reconfiguration of the polarity pattern to suit the new treatment volume.

34. A method in accordance with claim 33, wherein said reconfiguration is based on the matching of the profile of the treatment volume with a catalogue of pre-established treatment volumes and corresponding polarity patterns.
